(19) United States
(12) Patent Application Publication
(10) Pub. No.: US 2020/0197311 A1
(43) Pub. Date: Jun. 25, 2020

(54) AMORPHOUS NANOSTRUCTURED PHARMACEUTICAL MATERIALS

(71) Applicant: Novartis AG, Basel (CH)

(72) Inventors: Daniel HUANG, Palo Alto, CA (US); Dierk WIECKHUSEN, Basel (CH); Danforth MILLER, San Carlos, CA (US)

(21) Appl. No.: 16/620,757

(22) PCT Filed: Jun. 11, 2018
(86) PCT No.: PCT/IB2018/054201

§ 371 Date: Dec. 9, 2019

Related U.S. Application Data
(60) Provisional application No. 62/518,126, filed on Jun. 12, 2017.

(51) Int. Cl. A61K 9/16; A61K 9/00

(52) U.S. Cl. CPC A61K 9/1694; A61K 9/0053; A61K 9/007

(57) ABSTRACT

Embodiments of the invention relate to a process for enhancing the bioavailability of poorly soluble active ingredients, and to formulations of powders made by such process. Embodiments of the invention comprise a spinodal decomposition method by which low, sparingly or poorly-soluble materials are converted to amorphous materials with, improved or enhanced solubility suitable for therapeutic use. The powder formulations are useful for the treatment of diseases and conditions, especially respiratory diseases and conditions.

### FIELD OF THE INVENTION

The invention relates to a process for enhancing the bioavailability of poorly soluble active ingredients, and to formulations of powders made by such process. Embodiments of the invention comprise a spinodal decomposition method by which low, sparingly or poorly-soluble materials are converted to amorphous nanostructured materials with improved or enhanced solubility suitable for therapeutic use. The powder formulations are suitable for administration by a variety of means, and useful for the treatment of diseases and conditions, such as respiratory diseases and conditions.

### BACKGROUND

An increasing number of developmental new chemical entities (NCEs) have poor aqueous solubility, which has led to exploration of effective means to overcome their low bioavailability as a consequence of poor solubility. Poorly water-soluble drugs show a number of negative clinical effects, such as high local drug concentrations at sites of aggregate deposition, which could be associated with local toxic effects of the drug and decreased bioavailability. It is estimated that 25 to 40% of the already known, as well as a high percentage of newly developed drug substances, exhibit poor solubility characteristics and thus present a major problem in pharmaceutical formulations.

The solubility issues complicating the delivery of existing and new drugs have generated significant efforts in formulation and process development. Various traditional techniques which have been used for solubility enhancement of BCS Class II and IV drugs include use of micronization, co-solvents, amorphous forms, chemical modification of drug, use of surfactants, inclusion complexes, use of hydrates or solvates, use of soluble prodrugs, application of ultrasonic waves, functional polymer technology, controlled precipitation technology, evaporative precipitation in aqueous solution, selective adsorption on insoluble carriers. Novel drug delivery technologies developed in recent years for solubility enhancement of insoluble drugs include nanosizing technologies, lipid-based delivery systems, micellar technologies, porous micro particle technologies, hot-melt extrusion, and solid dispersion technique.

The above listed technologies have many drawbacks including complicated procedures and processing equipment, difficulty in controlling the key properties such as particle size and morphology, and requiring multiple excipients for processability and stability.

### SUMMARY OF THE PRESENT INVENTION

Accordingly, in embodiments of the present invention, there is provided a simple process to produce amorphous nanostructured pharmaceutical material for therapeutic uses.

In embodiments of the present invention, there is provided a simple process to produce amorphous nanostructured material without using any excipients.

Embodiments of the invention therefore comprise a method for preparing an amorphous nanostructured material, the method comprising: preparing a suspension or dispersion of a poorly water-soluble starting material in a solvent (or a solvent system), heating said suspension or dispersion to a temperature sufficient to dissolve the starting material thereby forming an intermediate solution; quenching said intermediate solution in a sink condition (to result in a spontaneous or near spontaneous liquid-liquid phase separation which then yields a first material-rich phase and a second solvent-rich phase; and mixing, using a high shear mixing apparatus until a generally or substantially homogenous mixture is obtained; and collecting said solid particles. Normally, in the spinodal decomposition process, phase separation is nearly instantaneous, but particle formation is not instantaneous. Therefore, the process may include an optional step of allowing the quenched formulation to dwell for a period of time to permit coarsening of material-rich droplets and formation of solid particles of the material. In embodiments of the invention, the suspension or dispersion comprises only the starting material and solvent. In embodiments of the invention, the quenching may further comprise metering the intermediate solution into a quench substrate or matrix. In embodiments of the invention, the starting material may be a pharmaceutically active material.

In embodiments of the present invention, amorphous nanostructured pharmaceutical materials are obtained when a heated solution containing drug substance is quenched into a quench matrix or substrate under high-shear mixing.

Embodiments of the present invention comprise particles having a uniform particle size distribution and which provide superior control of both dissolution and solubility of a drug substance.

Embodiments of the present invention comprise primary particles having a smallest dimension of about 100-500 nanometers, and agglomerates of primary particles of about 1-20 microns. In general, nanostructured materials are those with a structure in which the dominant or characteristic length scale is on the order of one to a few hundred nanometers. This gives these materials a greater specific surface area and/or a smaller radius of curvature than ordinary (e.g. non-nano structured) materials, enhancing properties such as dissolution rate and solubility.

Embodiments of a formulation and a process of the present invention afford the formation of amorphous nanostructured pharmaceutical material in a single step without using any carriers such as polymers, surfactants, porous silica, etc. The resulting new form of drug substance exhibits increased dissolution rate as well as improved solubility (compared to the original form of drug substance) which leads to higher bioavailability.

Embodiments of particles made by embodiments of the formulation and process of the present invention retain a high degree of physical and chemical stability. Because no excipients are required, embodiments comprising “pure” active pharmaceutical ingredient is easily formulated for a variety of applications.

Embodiments of the present invention are suitably formulated as medicaments for oral delivery.

Embodiments of the present invention are suitably formulated as particles for inhalation. Aspects of such inhalation particles comprise respirable agglomerates of nanoparticles, wherein the respirable agglomerates have a maximum geometric dimension (e.g., a diameter) of 1-10 microns, such as 2-5 microns.

Embodiments of the invention comprise an integrated process for obtaining amorphous nanostructured particles which particles are then initiated into an emulsion-based spray-drying particle engineering process (e.g., PulmoSphere). An exemplary PulmoSphere particle engineering process is described in U.S. Pat. Nos. 6,565,885, 8,168,223, and 8,349,294. Advantageously, in embodiments of the invention wherein tetrahydrofuran (THF) is used as the solvent for the API, the use of THF in the spinodal decomposition process does not interfere with the PulmoSphere emulsion.

Embodiments of the present invention comprise a two-step, integrated process for making amorphous nanostructured particles and formulating them as engineered particles for inhalation.

In embodiments of the invention, certain steps may be combined. For example mixing may be combined with the quenching. This has the advantage of speeding the timescale of mixing, which in turn facilitates the desired nanoscale geometry. Two timescales are important in such processes: the mixing time for the two solvents and the overall precipitation time of the drug. This ratio of timescales is a dimensionless quantity known as the Damkohler number, Da. Reduction of the mixing time to a value less than the precipitation time (Da<1) results in uniform mixing and a smaller, more uniform particle size distribution.

In in embodiments of the invention, low Damkohler numbers may be achieved by hardware design to reduce the mixing time (via shear forces, turbulent flow, high gravity, etc.) or by formulation design to increase the precipitation time.

In embodiments of the invention, a quenching feed rate is that which is slow enough to allow the spinodal process to take place. Functionally, the quenching feed rate (or metering rate) should be slow or gradual enough such that the metering liquid experiences a constant temperature environment. Put another way, the hot solution should not be added rapidly enough such that it creates a significant local temperature change in the quench solution. In embodiments of the invention, the forgoing comprises a sink condition; that is, all of the hot liquid experiences the same temperature. For example, when using ice water as the quenching medium, a desired sink condition is the maintenance of 0° C. In some embodiments, a liquid feed rate is 0.1 to 1 mL per minute. In embodiments of the invention, mixing may be at a shear rate of 4000 to 14,000 s⁻¹.

Embodiments of the invention comprise a method for preparing an amorphous active material, such as an active pharmaceutical ingredient, the method comprising preparing a suspension or dispersion of an active in a solvent (or a solvent system system), wherein the suspension or dispersion comprises only the active (which can be a single active or two or more actives in combination) and solvent; heating said suspension or dispersion to a temperature sufficient to dissolve the active ingredient(s); metering this solution at a controlled rate into a temperature-controlled quenching medium under high-shear mixing conditions to result in a spontaneous liquid-liquid phase separation, resulting in a first active-rich phase and a second solvent-rich phase; (optionally) allowing the quenched formulation to dwell for a period of time to permit coarsening of drug rich droplets and precipitation thereof into solid particles; and collecting said solid particles. In embodiments of the invention, the solid particles thus collected from the spinodal decomposition process may be further formulated as an engineered particle, such as a PulmoSphere engineered particle.

Embodiments of the invention comprise any of the foregoing wherein the material comprises a drug, active ingredient, active agent, or any therapeutic or nutraceutical material.

It is noted that the collection step is intended to be a functional definition and is not to be considered as limited to a particular process or apparatus. Collection may comprise a single step, or multiple steps. By way of nonlimiting examples, particles can be collected by physical force, such as by gravitational separation. Particles can be isolated by physical process, such as by solvent removal. Solvent removal, in turn, may comprise a variety of processes as known to the art for example: spray drying, freeze-drying, spray freeze-drying, supercritical processes, etc.

### Terms

Terms used in the specification have the following meanings:

“Active”, “active ingredient”, “therapeutically active ingredient”, “active agent”, “drug” or “drug substance” as used herein means the active ingredient of a pharmaceutical, also known as an active pharmaceutical ingredient (API).

“Amorphous” as used herein refers to a state in which the material lacks long-range order at the molecular level and, depending upon temperature, may exhibit the physical properties of a solid or a liquid. Upon heating, a change from solid to liquid properties occurs at the “glass transition” temperature, Tg.

“Bulk density” is defined as the mass of a granular material divided by its macroscopic volume, and is measured by simply pouring the granular material into a cavity of known volume without using any additional force (e.g., tapping or shaking).

“Crystalline” as used herein refers to a solid phase in which the material has a regular ordered internal structure at the molecular level and gives a distinctive X-ray diffraction pattern with defined diffraction peaks. Such materials when heated sufficiently will also exhibit the properties of a liquid, but the change from solid to liquid is characterised by a phase change, typically first order (“melting point”). In the context of the present invention, a crystalline active ingredient means an active ingredient with crystallinity of greater than 85%. In certain embodiments the crystallinity is suitably greater than 90%. In other embodiments the crystallinity is suitably greater than 95%.

“Drug Loading” as used herein refers to the percentage of active ingredient(s) on a mass basis in the total mass of the formulation.

“Mass median diameter” or “MMD” or “×50” as used herein means the median diameter of a plurality of particles, typically in a polydisperse particle population, i.e., consisting of a range of particle sizes. MMD values as reported herein are determined by laser diffraction (Sympatec Helos, Clausthal-Zellerfeld, Germany), unless the context indicates otherwise. d_{g} represents the geometric diameter of a single particle.

“Tapped densities” or ρ_{tapped}, as used herein were measured according to Method I, as described in USP <616>. Tapped densities represent an approximation of particle density, with measured values that are approximately 20% less than the actual particle density. Tapped density may be measured by placing the material in a sample cell, tapping the material, and adding additional material to the sample cell until it is full and no longer densifies upon further tapping.

“Median aerodynamic diameter of the primary particles” or Dₐ as used herein, is calculated from the mass median diameter of the bulk powder as determined via laser diffraction (×50) at a dispersing pressure sufficient to create primary particles (e.g., 4 bar), and their tapped density, namely: Dₐ=×50 (ρ_{tapped})^{1/2}.

“Delivered Dose” or “DD” as used herein refers to an indication of the delivery of dry powder from an inhaler device after an actuation or dispersion event from a powder unit. DD is defined as the ratio of the dose delivered by an inhaler device to the nominal or metered dose. The DD is an experimentally determined parameter, and may be determined using an in vitro device set up which mimics patient dosing. DD is also sometimes referred to as the emitted dose (ED).

“Mass median aerodynamic diameter” or “MMAD” as used herein refer to the median aerodynamic size of a plurality of particles, typically in a polydisperse population. The “aerodynamic diameter” is the diameter of a unit density sphere having the same settling velocity, generally in air, as a powder and is therefore a useful way to characterize an aerosolized powder or other dispersed particle or particle formulation in terms of its settling behaviour. The aerodynamic particle size distributions (APSD) and MMAD are determined herein by cascade impaction, using a Next Generation Impactor™. In general, if the particles are aerodynamically too large, fewer particles will reach the deep lung. If the particles are too small, a larger percentage of the particles may be exhaled. In contrast, dₐ represents the aerodynamic diameter for a single particle.

“Primary particles” refer to the smallest divisible particles that are present in an agglomerated bulk powder. The primary particle size distribution is determined via dispersion of the bulk powder at high pressure and measurement of the primary particle size distribution via laser diffraction. A plot of size as a function of increasing dispersion pressure is made until a constant size is achieved. The particle size distribution measured at this pressure represents that of the primary particles.

“Sink condition” unless otherwise clear from the context, means the use of a volume and temperature of quench solution, such that the heated suspension or dispersion of API dissolved in a solvent or a multiple solvent system experiences a substantially constant quench temperature environment.

Throughout this specification and in the claims that follow, unless the context requires otherwise, the word “comprise”, or variations such as “comprises” or “comprising”, should be understood to imply the inclusion of a stated integer or step or group of integers or steps but not the exclusion of any other integer or step or group of integers or steps.

The entire disclosure of each United States patent and international patent application mentioned in this patent specification is fully incorporated by reference herein for all purposes.

### DESCRIPTION OF THE DRAWINGS

The dry powder formulation of the present invention may be described with reference to the accompanying drawings. In those drawings.

FIG. 1 is an idealized state diagram of temperature and free energy versus concentration fraction showing the binodal boundary and spinodal region.

FIG. 2 is a scanning electron microscope (SEM) image of a neat (excipient-free) drug substance (hereinafter referred to as drug Z) powder made in accordance with embodiments of the present invention showing amorphous nanostructured particles, and showing the results in material with a honeycomb morphology with interstitial spaces (pores).

FIG. 3 is a scanning electron microscope (SEM) image of a spray-dried drug substance (drug Z) powder made in accordance with embodiments of the present invention showing a desirable morphology. This image shows the result of the integrated spinodal process whereby the neat drug substance particles are embedded within a PulmoSphere engineered particle matrix.

FIG. 4 is a graph of amount dissolved (mg/mL) over time (minutes) for different formulations of a spray-dried drug substance powder. Two formulations (designation 123-32-3—curve labelled with a square; and 123-32-6—curve labelled with a diamond) are conventional spray-dried engineered particle formulations The curve labelled with a triangle represents a formulation (designation 123-32-1) of neat active made in accordance with embodiments of the present invention. A micronized crystalline control (“neutral form” or “NX’, labelled with an “x”) is supplied for comparative purposes.

FIG. 5 is a diagrammatic illustration of a general process according to embodiments of the present invention.

FIG. 6 is a diagrammatic illustration of a process according to embodiments of the present invention.

FIG. 7 shows XRPD patterns of a neat drug substance (drug Z) powder made in accordance with embodiments of the present invention showing two different lots of resulting amorphous nanoparticles, and comparing with an XRPD pattern of conventional crystalline drug Z. The plot is of intensity versus two theta (degrees).

FIGS. 8A and 8B are SEM images of a drug substance (drug Z) powder made in accordance with embodiments of the present invention showing that the majority of primary particles are between 200 and 300 nm (0.2-0.3 microns) in size with the larger particles 1 to 2 μm in size. Particle size is fairly uniform, as shown particularly by the image in FIG. 8B, which is the same image as FIG. 8A but at twice the magnification.

FIGS. 9A and 9B are SEM images of a spray-dried (drug Z) powder made in accordance with embodiments of the present invention showing powders manufactured by the integrated spinodal PulmoSphere process wherein an insoluble material is first subjected to the spinodal process to render it soluble, and then is made into an inhalation particle using the PulmoSphere engineered particle technology.

FIG. 10 is a near infrared spectroscopy plot showing crystalline API, neat API made by a spinodal process of the present invention; a lot of powder made by an integrated spinodal process of the present invention; and a lot of excipient (DSPC) placebo powder. The Figure demonstrates the amorphous nature of a formulated drug made by embodiments of the present invention comprising both the spinodal process and the PulmoSphere particle engineering process. A crystalline drug is shown for comparison. The X-axis of FIG. 10 (wave number) is labelled from 6966 to 6410 cm⁻¹, while the Y-axis (absorption) is labelled from 0.11 to 0.18.

FIG. 11A is an in-vitro concentration versus time plot showing dissolution profiles of a micronized crystalline form of drug Z, and a spray-dried amorphous form manufactured using the processes described in Example 2. The plot shows a high rate of dissolution for the amorphous form. FIG. 11B shows pharmacokinetic profiles (in vivo rat study) of the same crystalline form of drug Z and the amorphous form according to Example 2. The half-life of the amorphous nanostructured form is markedly shorter (approximately 5 hours) than that of its crystalline counterpart, indicating faster dissolution and absorption of the amorphous nanostructured form of the present invention.

### DETAILED DESCRIPTION

Embodiments of the present invention are directed to a formulation and process to preparing an amorphous nanostructured active material comprising preparing a suspension or dispersion of a poorly water-soluble active material in a solvent, wherein the solvent is selected to solubilize a desired quantity of the material upon heating, and wherein the suspension or dispersion comprises the active material and solvent; heating said suspension or dispersion to a temperature sufficient to dissolve the active material to yield a solution; quenching the solution by metering into a temperature-controlled quenching medium while mixing using high-shear, resulting in a spontaneous liquid-liquid phase separation, yielding a first active material-rich phase and a second solvent-rich phase wherein solid amorphous particles of active material precipitate from the first active material-rich phase; and collecting said solid amorphous particles.

Embodiments of the present invention are directed to a formulation and process to preparing an amorphous nanostructured pharmaceutically active material comprising preparing a suspension or dispersion of a poorly water-soluble pharmaceutically active material in a solvent, wherein the solvent is selected to solubilize a desired quantity of the active material upon heating, and wherein the suspension or dispersion comprises the pharmaceutically active material and solvent; heating said suspension or dispersion to a temperature sufficient to dissolve the active material to yield a solution; quenching the solution by metering into a temperature-controlled quenching medium while mixing using high-shear, resulting in a spontaneous liquid-liquid phase separation, yielding a first active material-rich phase and a second solvent-rich phase wherein solid amorphous particles of pharmaceutically active material precipitate from the first active material-rich phase; and collecting said solid amorphous particles comprising the pharmaceutically active material. Optionally, the quenched solution is allowed to dwell for a period of time to permit coarsening of drug rich droplets and precipitation thereof into solid particles.

Embodiments of the present invention are directed to a formulation and process for preparing a pharmaceutical powder comprising preparing a suspension or dispersion of a poorly-water soluble active pharmaceutical ingredient in a solvent, wherein the suspension or dispersion consists of only the material and solvent; heating said suspension or dispersion to a temperature sufficient to dissolve the active pharmaceutical ingredient to yield a solution; quenching the solution, by metering into a temperature-controlled quenching medium while mixing using high-shear, resulting in a spontaneous liquid-liquid phase separation, yielding a first active-rich phase and a second solvent-rich phase; and allowing the quenched formulation to dwell to permit coarsening of active-rich droplets and precipitation thereof into solid nanoparticles of substantially pure active pharmaceutical ingredient in amorphous form; collecting said solid particles; preparing an emulsion of the solid nanoparticles of active pharmaceutical ingredient in a solvent or suspending agent, together with a phospholipid to yield a feedstock; and spray drying feedstock to yield nanoparticles of active pharmaceutical ingredient comprising a honeycomb morphology with interstitial spaces.

### Formulation/Particle Engineering

Embodiments of the invention comprise methods and materials for preparing amorphous nanostructured pharmaceutical suspensions or dispersions.

Embodiments of methods employ a thermal quenching process coupled with a high-shear mixing procedure to form particles with an amorphous nano-scaled honeycomb morphology with interstitial spaces. Thermal quenching is a process by which a solution of one or more components can separate into distinct regions (or phases) of different chemical composition and physical properties.

Embodiments of the invention comprise a process whereby a crystalline substance which is poorly soluble in aqueous media can be converted into amorphous nanoparticles, resulting in a significant increase in the dissolution rate and solubility

Embodiments of the invention comprise a process whereby a sparingly aqueous soluble substance can be converted to one having greater in solubility, such as 2-30 times greater, or 5-20 times greater, or 6-10 times greater.

Embodiments of the invention comprise a product whereby a sparingly aqueous soluble substance can be converted to one having greater in solubility, such as 2-30 times greater, or 5-20 times greater, or 7-10 times greater.

Embodiments of the invention comprise a process whereby a starting substance having an initial percentage dissolved of less than 20% can be converted to one having a percentage dissolved of 60% or 70% or 80% or 90% or 95% or more.

Embodiments of the invention comprise a product having a percentage dissolved of 60% or 70% or 80% or 90% or 95% or more.

Embodiments of the present formulation and process allow the formation of an amorphous nanostructured material, for example a pharmaceutically active material, in a single step without using any excipients such as polymers, surfactants, porous silica, etc. Such amorphous nanostructured pharmaceutical materials have increased dissolution rates, as well as improved solubility (compared to the original crystalline drug substance) which may lead to higher bioavailability. Embodiments of the present invention comprising amorphous nanostructured materials retain a high degree of physical and chemical stability. In embodiments of the present invention wherein the material is a pharmaceutical material and wherein excipients are not used, the resultant “pure” or “neat” active pharmaceutical ingredient is easily formulated for a variety of applications.

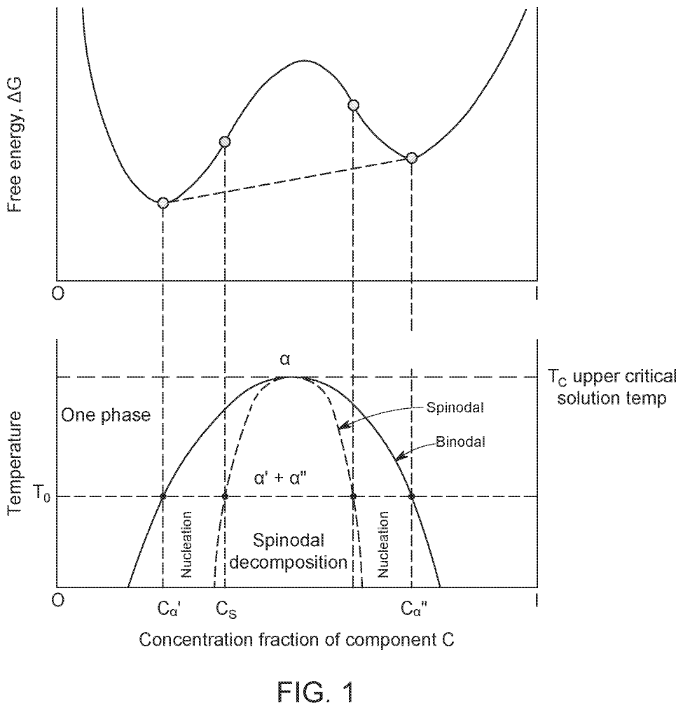

Spinodal decomposition is a process by which a solution of two or more components can separate into distinct regions (or phases) of different chemical composition and physical properties. As shown in FIG. 1, phase separation may occur whenever a material is within the thermodynamically unstable region of the phase diagram. The boundary of this unstable region (the binodal) is defined by a common tangent of the thermodynamic potential. Inside the binodal boundary, the spinodal region is entered when the curvature of the Gibbs free energy becomes negative. The binodal and spinodal meet at a critical point—the Upper Critical Solution Temperature (UCST). Spinodal decomposition occurs when a material is brought into the spinodal phase region. The phase separation proceeds through spinodal decomposition (unstable region) or nucleation and growth (metastable region) followed by a coarsening process. Generally, to reach the spinodal region of the phase diagram, the system must be brought through the binodal region, where nucleation may occur. Because nucleation is undesirable, spinodal decomposition requires a very fast transition (a quench) to quickly bring the system from the stable region through the meta-stable nucleation region and well into the mechanically unstable spinodal phase region. In general, the spinodal decomposition process has the following characteristics: (i) it occurs spontaneously when the composition is within the spinodal region; (ii) it is controlled by thermodynamics and/or kinetics; (iii) phase boundaries are diffuse; and (iv) the material forms an interconnected structure.

In the spinodal decomposition process, the homogeneous solution containing dissolved solute (for example drug Z) phase separates into a solute-rich phase and a solvent-rich (solute-lean) phase upon quenching. Above the critical composition of solute, the solute-rich phase first forms a continuous wave stream. As the amplitude of the wave increases, it breaks into droplets facilitated by a high-shear flow field in the continuous phase. The solute-lean phase is composed of nearly pure diluent, so it is easily mixed with the rest of continuous phase to form a single solvent phase. The solvent in the solute-rich droplets diffuses to the continuous phase and solid particles are formed when the solute reaches its (amorphous) solubility limit. Because the droplets form solid particles, size control of the droplet is a critical step for regulating the final solid particle size. To control the droplet formation during the spinodal decomposition process, it is important to understand the phase separation process caused by the UCST-type phase behavior, the kinetics of the fluid flow field, and the influence of the growth process of droplets after the phase separation.

In the spinodal decomposition process, an initial phase transformation tends to be fast, on the order of a few milliseconds. For two liquid phases separating from one miscible liquid phase, experiments have demonstrated that following the initial separation, micro-domains grow by diffusion and coalescence. The later stage of a spinodal decomposition phase transition of a liquid mixture involves coarsening of the phase-separated droplets. During this stage, the effect of hydrodynamic interactions on droplets dominates the surface tension forces; droplets in the system coalesce and/or break up under the influence of inertial and viscous forces. Thus the mechanisms that control the spinodal decomposition phase transition depend not only on the thermodynamics but also on the process. As a result, the process and the competing mechanisms underlying the phase transition must both be considered when preparing a spinodal decomposition suspension. This suspension may be dried to yield solid, spinodal particles, or may be used in downstream processing as part of a particle engineering process, for example, a PulmoSphere process. When used as part of a downstream particle engineering process a suspension or dispersion of nanostructured amorphous particles resulting from the spinodal decomposition process of the present invention is sometimes referred to herein as the “annex suspension”. In other words, if intended to be further processed into engineered particles, the annex suspension comprises the amorphous structured nanoparticles suspended or dispersed in the quench solution, such as cold water. The uniformity achieved by the spinodal decomposition method described herein can be beneficial in downstream particle engineering processes, such as the suspension-based PulmoSphere process and/or a carrier-based process, for example, the iPulmoSphere process.

In embodiments of a process of the present invention, a spontaneous liquid-liquid phase separation occurs to form drug-rich and solvent-rich phases. The formation of droplet size is directly related to the final particle size. Accordingly, in embodiments of the present invention, preparation conditions comprise feedstock feed rate, mixing shear rate, temperature difference between feedstock and quenching medium, initial drug concentration, selection of solvent system, and quenching temperature.

The temperature differential between the temperature of the initial feedstock and the quenching medium is determined empirically, by quenching deep in the two-phase region defining the spinodal region, in other words as far away as practicable from the two-phase region bounding the spinodal. A temperature above T_{c} is determined experimentally by ensuring there is no, or essentially no, or a desired minimum of, insoluble material at whatever drug loading is desired.

In embodiments of the invention, high-shear mixing is used to keep the phase separation zone in an isothermal and homogeneous environment. With the passage of time, the drug-rich droplets can grow through a process by coarsening. The effect of the coarsening process, which is induced by differential interfacial tension in the liquid-liquid phase separation domains, is considered to play an important role in determining the final morphology. It may be noted that the coarsening process results in an amorphous nanostructure primarily via one or more of: Ostwald ripening, coalescence, or hydrodynamic flow mechanisms. Thus, the coarsening process should be considered a kinetic parameter to control the morphology of the resultant amorphous nanostructure.

In embodiments of the invention, therefore two general processes apply to the formation of particles: the thermodynamics of quenching, and the kinetics of feed rate and mixing.

In embodiments of the present invention, process conditions comprise those which relatively quickly effect heat transfer between the feedstock and quench solution. This results in a favorable nano-scale structure as the droplet growth is rapidly arrested upon quenching the solution.

In embodiments of the present invention, in a first step, a hydrophobic drug substance with low water solubility is dissolved in a solvent or a solvent system at an elevated temperature (for example 60-90° C.). In embodiments of the present invention, the solvent may comprise water. In embodiments of the present invention, the solvent system may comprise one or more water-miscible solvents. In some embodiments, the solvent system may comprise tetrahydrofuran and water. In some embodiments the tetrahydrofuran and water is present in an 80:20 w/w ratio. In a second step, the heated solution with dissolved drug substance is gradually metered (for example at 0.1 to 2 mL/min) into a quenching medium or heat transfer material. In embodiments of the invention, the quenching medium comprises a cold-water bath, for example ice water (at 0° C.). In embodiments of the invention, the quenching medium comprises one which is miscible with the initial solvent(s) that is that used to dissolve the active ingredient, yet is a nonsolvent or poor solvent for the active.

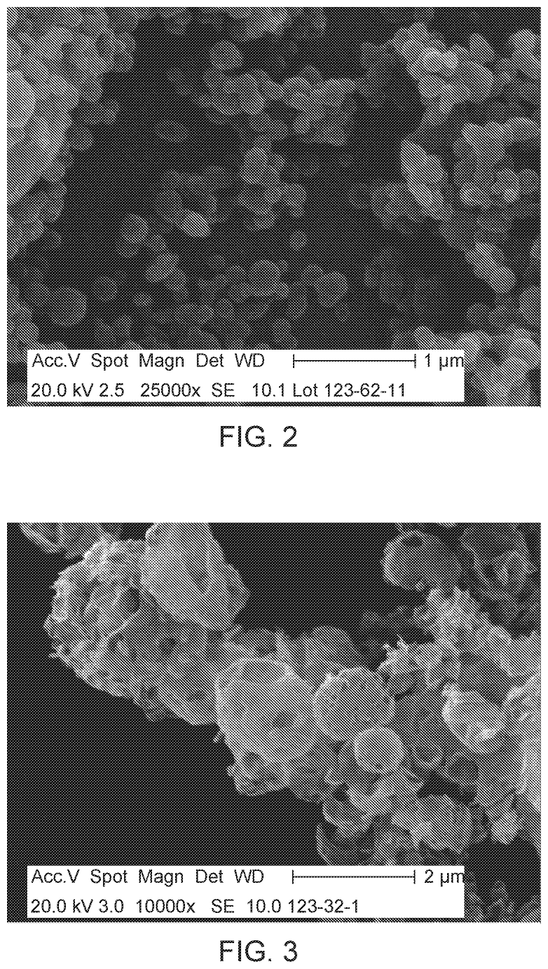

During the quenching of hot solution containing dissolved API in the quenching medium, mixing may be employed to allow formation of the resulting solid in a well-mixed environment. In some embodiments, the mixing may comprise high-shear mixing, for example at a shear rate of about 2000 s⁻¹ or greater. Due to the low solubility of drug substance in excess cold water, precipitation of the API is a function of both temperature drop and from solvent diffusion. At API precipitation is complete, the resultant amorphous nanostructured material (shown by SEM in FIG. 2) shows uniformity of particle size, indicative of an orderly phase transformation. That is to say that by control of process conditions, specifically including the sink condition and feed rate, one achieves an orderly phase transition through the spinodal process, which yields generally uniformly sized nanoparticles.

In aspects of the invention, a material feed rate may be from 0.1 to 1 mL/min, and preferably 0.2 to 0.8, or 0.3 to 0.5 mL/min. A mixing shear rate may be 2000 to 18,000 s⁻¹, such as 6000 to 12000 s⁻¹.

FIG. 3 shows engineered particles made by the integrated spinodal process is described herein, wherein the particles exhibit a honeycomb morphology with interstitial spaces (pores). Such a honeycomb morphology is a function of controlling the process conditions, e.g., metering rate and sink conditions, which in embodiments of the invention, results in this type of morphology. By control of process conditions, in embodiments of the invention modifications of the morphology may be obtained.

### The Active Agent

The active agent(s) described herein may comprise an agent, drug, compound, composition of matter or mixture thereof which provides some pharmacologic, often beneficial, effect. As used herein, the terms further include any physiologically or pharmacologically active substance that produces a localized or systemic effect in a patient. An active agent for incorporation in the pharmaceutical formulation described herein may be an inorganic or an organic compound, including, without limitation, drugs which act on: the peripheral nerves, adrenergic receptors, cholinergic receptors, the skeletal muscles, the cardiovascular system, smooth muscles, the blood circulatory system, synoptic sites, neuroeffector junctional sites, endocrine and hormone systems, the immunological system, the reproductive system, the skeletal system, autacoid systems, the alimentary and excretory systems, the histamine system, and the central nervous system. Suitable active agents may be selected from, for example, hypnotics and sedatives, tranquilizers, respiratory drugs, drugs for treating asthma and COPD, anticonvulsants, muscle relaxants, antiparkinson agents (dopamine antagnonists), analgesics, anti-inflammatories, antianxiety drugs (anxiolytics), appetite suppressants, antimigraine agents, muscle contractants, anti-infectives (antibiotics, antivirals, antifungals, vaccines) antiarthritics, antimalarials, antiemetics, anepileptics, bronchodilators, cytokines, growth factors, anti-cancer agents, antithrombotic agents, antihypertensives, cardiovascular drugs, antiarrhythmics, antioxicants, anti-asthma agents, hormonal agents including contraceptives, sympathomimetics, diuretics, lipid regulating agents, antiandrogenic agents, antiparasitics, anticoagulants, neoplastics, antineoplastics, hypoglycemics, nutritional agents and supplements, growth supplements, antienteritis agents, vaccines, antibodies, diagnostic agents, and contrasting agents. The active agent, when administered by inhalation, may act locally or systemically.

The active agent may fall into one of a number of structural classes, including but not limited to small molecules, peptides, polypeptides, antibodies, antibody fragments, proteins, polysaccharides, steroids, proteins capable of eliciting physiological effects, nucleotides, oligonucleotides, polynucleotides, fats, electrolytes, and the like.

In embodiments of the invention, the active agent may include or comprise any active pharmaceutical ingredient that is useful for treating inflammatory or obstructive airways diseases, such as asthma and/or COPD. Suitable active ingredients include long acting beta 2 agonist, such as salmeterol, formoterol, indacaterol and salts thereof, muscarinic antagonists, such as tiotropium and glycopyrronium and salts thereof, and corticosteroids including budesonide, ciclesonide, fluticasone, mometasone and salts thereof. Suitable combinations include (formoterol fumarate and budesonide), (salmeterol xinafoate and fluticasone propionate), (salmeterol xinofoate and tiotropium bromide), (indacaterol maleate and glycopyrronium bromide), and (indacaterol and mometasone).

The amount of active agent in the pharmaceutical formulation will be that amount necessary to deliver a therapeutically effective amount of the active agent per unit dose to achieve the desired result. In practice, this will vary widely depending upon the particular agent, its activity, the severity of the condition to be treated, the patient population, dosing requirements, and the desired therapeutic effect. The composition will generally contain anywhere from about 1% by weight to about 100% by weight active agent, typically from about 2% to about 95% by weight active agent, and more typically from about 5% to 85% by weight active agent, and will also depend upon the relative amounts of additives contained in the composition. In embodiments of the invention, compositions of the invention are particularly useful for active agents that are delivered in doses of from 0.001 mg/day to 10 g/day, such as from 0.01 mg/day to 1 g/day, or from 0.1 mg/day to 500 mg/day. It is to be understood that more than one active agent may be incorporated into the formulations described herein and that the use of the term “agent” in no way excludes the use of two or more such agents.

In embodiments of the present invention, the poorly soluble starting material which is made into a more soluble, amorphous nanoparticle or nanoparticle aggregate may be other than a pharmaceutical active ingredient. For example, the material may be a placebo.

The different free energies associated with each physical form gives rise to measurable differences in physical properties. The free energy-temperature diagram shown in FIG. 1 illustrates the bimodal and spinodal phase boundaries for a single-component (solute/solvent) system. In the figure, T_{c} is the upper critical solution temperature, that is, the temperature at which all or substantially all solids are dissolved into a single-phase, homogenous system. The lower dashed line is the T₀ point, that is the quenched temperature, and the difference between T_{c} and T₀ is the temperature differential. The dashed parabola encloses the spinodal region.

Because of the high internal energy, amorphous solids generally have a higher kinetic solubility and dissolution rate. The concept of solubility implies that the process of solution has reached an equilibrium state such that the solution has become saturated. The intrinsic solubility of a substance depends on the particular solid phase that is present. Since free energies of physical forms are responsible for the difference in solubilities and dissolution rates, the largest difference in solubility is observed between amorphous and crystalline materials. Equation I below depicts the ratio of solubility between amorphous and crystalline materials related to the free energy difference at specific temperature.

$$\frac{Sa}{Sc} \approx exp ( \frac{Δ G}{R \cdot T} ) \qquad ( Equation I )$$

Where Sa is the solubility of amorphous and Sc is the solubility of crystalline materials, ΔG is Gibbs free energy difference, R is the universal gas constant, and T is absolute temperature.

It has been reported that the solubility ratio between polymorphic pairs is generally less than two, although in certain cases, higher ratios are observed. In the simplest form, differences in solubility are a reflection of the free energy differences between polymorphs. In embodiments of the invention, solubility of the amorphous form can range from two times to thirty times the solubility of the crystalline form. Thus products and processes of the present invention may possess significantly greater solubility compared to crystalline forms.

Alternatively or additionally, poorly-water-soluble drugs maybe formulated as nano-scale drug particles. These nano-formulations offer increased dissolution rates for drug compounds and complement other technologies used to enhance bioavailability of insoluble compounds (BCS Class II and IV) such as solubility enhancers (i.e., surfactants), liquid-filled capsules or solid dispersions of drugs in their amorphous state. The advantages of nano-formulations in drug delivery have been demonstrated in vitro in dissolution testing and in vivo in both preclinical studies as well as clinical trials. The solid API dissolution rate is proportional to the surface area available for dissolution as described by the Noyes-Whitney equation:

dC/dt=A·D·((Cₛ−C)/d) Equation II

where dC/dt=dissolution rate, C is the concentration of drug in the medium at time t, A=particle surface area, D=diffusion coefficient, Cₛ=saturation solubility, d=effective boundary layer thickness.

According to this equation, the dissolution rate of a drug can be increased by: increasing surface area of the drug particle, increasing diffusivity which is difficult for a specific drug, improving apparent solubility of the drug under physiologically relevant conditions, and decreasing the diffusion layer thickness. Considering all these factors, decreasing particle size to the nanoscale offers an effective means to dramatically increase the surface area for a given quantity of material. Besides increased surface area, the percentage of molecules on the surface also increases. The use of products and processes of the present invention advantageously provide both nano-scale size and conversion to amorphous form in a unified process (i.e. a higher surface to volume ratio). Thus at least two distinct advantages flow from the present invention.

In addition to the dissolution rate enhancement described above, an increase in the saturation solubility of the nanosized API is also expected, as described by the Freundlich-Ostwald equation (Equation III):

Cₛ=C_{∞}·exp(2γM/rρRT) (Equation III)

where Cₛ=saturation solubility of the nanosized API, C_{∞}=saturation solubility of an infinitely large API crystal, γ is the particle-medium interfacial tension, M is the compound molecular weight, r is the particle radius, ρ is the density, R is the universal gas constant and T is the absolute temperature.

The key feature of this equation is that due to the effect of surface curvature, i.e., 1/r the saturation solubility would increase from a few percent up to 27% in solubility when particle size reduces to 10-100 nanometer range. This increase in saturation solubility leads to a further increase in dissolution rate and, as a result, nanosuspensions often achieve significantly higher exposure levels compared to conventional suspensions of micron-sized API. That is, when a pharmaceutical formulation made in accordance with the present invention is dosed into, for example tissue, blood or plasma, concentrations in the target organ are higher compared to such conventional preparations.

### Buffers/Optional Ingredients

Buffers are well known for pH control, both as a means to deliver a drug at a physiologically compatible pH (i.e., to improve tolerability), as well as to provide solution conditions favorable for chemical stability of a drug. In embodiments of formulations and processes of the present invention, the pH milieu of a drug can be controlled by co-formulating the drug and buffer together in the same particle.

Buffers or pH modifiers, such as histidine or phosphate, are commonly used in lyophilized or spray-dried formulations to control solution- and solid-state chemical degradation of proteins. Glycine may be used to control pH to solubilize proteins (such as insulin) in a spray-dried feedstock, to control pH to ensure room-temperature stability in the solid state, and to provide a powder at a near-neutral pH to help ensure tolerability. Preferred buffers include: histidine, glycine, acetate, and phosphate

Optional excipients include salts (e.g., sodium chloride, calcium chloride, sodium citrate), antioxidants (e.g., methionine), excipients to reduce protein aggregation in solution (e.g., arginine), taste-masking agents, and agents designed to improve the absorption of macromolecules into the systemic circulation (e.g., fumaryl diketopiperazine).

### Process

Following precipitation of particles, in some embodiments of the present invention spray drying is utilized to engineer particles for a specific purpose, such as particles for inhalation.

Embodiments of the present invention provide a process for preparing dry powder formulations for inhalation, comprising a formulation of spray-dried particles, the formulation containing at least one active ingredient that is suitable for treating obstructive or inflammatory airways diseases, particularly asthma and/or COPD.

Embodiments of the present invention provide a process for preparing dry powder formulations for inhalation, comprising a formulation of spray-dried particles, the formulation containing at least one active ingredient that is suitable for non-invasively treating diseases in the systemic circulation.

Spray-drying comprises four unit operations: feedstock preparation, atomization of the feedstock to produce micron-sized droplets, drying of the droplets in a hot gas, and collection of the dried particles with a bag-house or cyclone separator.

Embodiments of the process of the present invention comprise three steps, however in some embodiments two or even all three of these steps can be carried out substantially simultaneously, so in practice the process can in fact be considered as a single-step process. Solely for the purposes of describing the process of the present invention the three steps will be described separately, but such description is not intended to limit to a three-step process.

In embodiments of the present invention, a process of the present invention which yields dry powder particles comprises preparing a solution feedstock and removing solvent from the feedstock, such as by spray-drying, to provide the active dry powder particles.

In embodiments of the invention, the feedstock comprises at least one active dissolved in an aqueous-based liquid feedstock. In some embodiments, the feedstock comprises at least one active agent dissolved in an aqueous-based feedstock comprising an added co-solvent.

The particle formation process is highly complex and dependent on the coupled interplay between process variables such as initial droplet size, feedstock concentration and evaporation rate, along with the formulation physicochemical properties such as solubility, surface tension, viscosity, and the solid mechanical properties of the forming particle shell.

For amorphous solids it is important to control the moisture content of the drug product. The moisture content in the powder is preferably less than 5%, more typically less than 3%, or even 2% w/w. Moisture content must be high enough, however, to ensure that the powder does not exhibit significant electrostatic forces. The moisture content in the spray-dried powders may be determined by Karl Fischer titrimetry.

In some embodiments the feedstock is atomized with a twin-fluid nozzle, such as that described in U.S. Pat. Nos. 8,936,813 and 8,524,279. Significant broadening of the particle size distribution of the liquid droplets can occurs above solids loadings of about 1.5% w/w.

In some embodiments, narrow droplet size distributions can be achieved with plane film atomizers as disclosed for example in U.S. Pat. Nos. 7,967,221 and 8,616,464, especially at higher solids loadings. In some embodiments, the feedstock is atomized at solids loading between 0.1% and 10% w/w, such as 1% and 5% w/w.

Any spray-drying step and/or all of the spray-drying steps may be carried out using conventional equipment used to prepare spray dried particles for use in pharmaceuticals that are administered by inhalation. Commercially available spray-dryers include those manufactured by Büchi Ltd. and Niro Corp.

In some embodiments, the feedstock is sprayed into a current of warm filtered air that evaporates the solvent and conveys the dried product to a collector. The spent air is then exhausted with the solvent. Operating conditions of the spray-dryer such as inlet and outlet temperature, feed rate, atomization pressure, flow rate of the drying air, and nozzle configuration can be adjusted in order to produce the required particle size, moisture content, and production yield of the resulting dry particles. The selection of appropriate apparatus and processing conditions are within the purview of a skilled artisan in view of the teachings herein and may be accomplished without undue experimentation. Exemplary settings for a NIRO® PSD-1® scale dryer are as follows: an air inlet temperature between about 80° C. and about 200° C., such as between 110° C. and 170° C.; an air outlet between about 40° C. to about 120° C., such as about 60° C. and 100° C.; a liquid feed rate between about 30 g/min to about 120 g/min, such as about 50 g/min to 100 g/min; total air flow of about 140 scfm to about 230 scfm, such as about 160 scfm to 210 scfm; and an atomization air flow rate between about 30 scfm and about 90 scfm, such as about 40 scfm to 80 scfm. The solids content in the spray-drying feedstock will typically be in the range from 0.5% w/v (5 mg/ml) to 10% w/v (100 mg/ml), such as 1.0% w/v to 5.0% w/v. The settings will, of course, vary depending on the scale and type of equipment used, and the nature of the solvent system employed. In any event, the use of these and similar methods allow formation of particles with diameters appropriate for aerosol deposition into the lung.

Particles made in accordance with embodiments of the process of the present invention may be formulated to be delivered in a variety of ways, such as orally, transdermally, subcutaneously, intradermally, pulmonary, intraocularly, etc. In embodiments of the present invention, particles are prepared and engineered for inhalation delivery.

### Inhalation Delivery System

The present invention also provides a delivery system, comprising an inhaler and a dry powder formulation of the invention.

In one embodiment, the present invention is directed to a delivery system, comprising a dry powder inhaler and a dry powder formulation for inhalation that comprises spray-dried particles that contain a therapeutically active ingredient, wherein the in vitro total lung dose is between 60% and 100% w/w of the nominal dose, such as at least 65% or 70% or 75% or 80% or 85% of the nominal dose.

### Inhalers

Suitable dry powder inhaler (DPIs) include unit dose inhalers, where the dry powder is stored in a capsule or blister, and the patient loads one or more of the capsules or blisters into the device prior to use. Alternatively, multi-dose dry powder inhalers are contemplated where the dose is pre-packaged in foil-foil blisters, for example in a cartridge, strip or wheel. Formulations of the present invention are suitable for use with a broad range of devices, device resistances, and device flow rates. In embodiments of the invention, products and formulations of the present invention afford enhanced bioavailability.

### Aerosol Properties

The aerosol properties of the spray-dried powders using the integrated spinodal PulmoSphere formulation have essentially the same performance as that of the underlying PulmoSphere formulation. This is because the aerosol properties of PulmoSphere-based powders with embedded solids are dictated by the low-density and low-surface energy porous particles comprising the matrix. Low density or hollow particles are advantageous for several applications, but specifically for pulmonary drug delivery where they improve delivery efficiency by lowering the aerodynamic diameter of the particles. In addition, DSPC which is a low-surface energy material used in PulmoSphere formulations itself improves dispersibility and reduces interparticle cohesive forces as a means to maximize lung targeting, and enable improvements in the consistency of pulmonary drug delivery.

### Use in Therapy

Embodiments of the present invention provide a method for the treatment of an obstructive or inflammatory airways disease, especially asthma and chronic obstructive pulmonary disease, the method which comprises administering to a subject in need thereof an effective amount of the aforementioned dry powder formulation.

Embodiments of the present invention provide a method for the treatment of systemic diseases, the method which comprises administering to a subject in need thereof an effective amount of the aforementioned dry powder formulation.

### EXAMPLES

Drug Z is a potent and selective adenosine A2A receptor agonist which in vitro exhibits potent anti-inflammatory activity on a range of human cell types relevant to inflammatory respiratory diseases. drug Z exhibits efficacy in reduction of pulmonary inflammation in COPD, which may result in superior control of symptoms and exacerbations.

Drug Z is a high molecular weight, high polar surface area, and poorly soluble compound. Drug Z in its crystalline form is very insoluble in aqueous systems at physiological pH. In Example 1, crystalline drug Z drug substance was converted into amorphous nanoparticles by a spinodal decomposition thermal quenching process according to embodiments of the present invention, resulting in a significant increase in the dissolution rate and solubility from less than 20% to 80-100%.

### Example 1—Preparation of Spray-Dried Formulations of Neat API

Sufficient drug Z was first dissolved in a cosolvent system (75% w/w tetrahydrofuran and 15% w/w water) at an elevated temperature (65-70° C.) at a solids concentration of 2 w/w %. Then the heated solution with dissolved drug substance was gradually metered into an ice water bath (at 0° C.) which created a significant thermal gradient between drug solution and water bath. During the quenching of the hot solution containing dissolved API, high-shear mixing (about 8000 sec⁻¹) was used to enable solid formation in a well-mixed environment. Due to the low solubility of drug substance in excess cold water surroundings, precipitation took place both from the temperature drop as well as solvent diffusion. After completion of the precipitation process, the resultant amorphous nanostructured material had a honeycomb morphology with interstitial spaces (pores) (See FIG. 2).

In Example 2, amorphous nanoparticles made in accordance with Example 1 are formulated into engineered inhalation particles using a PulmoSphere formulation process.

### Example 2—Formulation of Amorphous Drug Z with PulmoSphere Process

Particles of amorphous drug Z, were produced by the spinodal process described in Example 1. The resulting particles, having an average primary particle size of 2.3 microns, and a bulk density of 0.22 g/cm³, were first suspended in water. This suspension was added to a PulmoSphere feedstock emulsion comprising 20% v/v PFOB in water stabilized by 90% w/w DSPC plus calcium chloride. This feedstock was then spray dried using a lab-scale spray dryer at an outlet temperature of 65-70 C The spray-dried particles (10% w/w drug Z/90% w/w DSPC/CaCl₂)) were collected using a cyclone collector. A particle yield was approximately 74%. Physical properties of the particles were tested and an average primary particle size was found to be 2.3 microns, with a bulk density of 0.22 g/cm³, and a water content of 3.5% w/w.

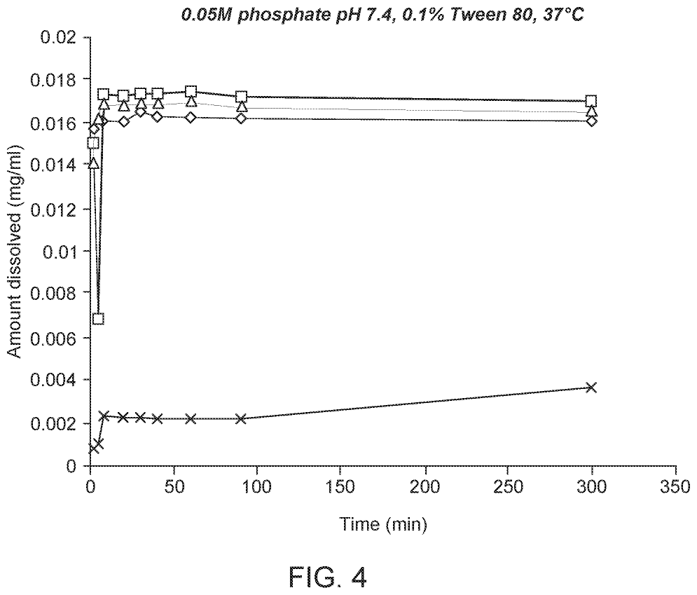

FIG. 4 shows dissolution profiles for this engineered particle formulation made in accordance with Example 2 (formulation 123-32-2—curve labelled with a triangle) compared to three comparative formulations. Comparative Formulation 123-32-3 (curve labelled with a square) is a non-spinodal, Pulmosphere engineered formulation wherein drug Z seed particles were first suspended in water, and mixed with an emulsion of 90% DSPC plus CaCl₂). PFOB (20% w/w) was mixed into the solution as the blowing agent. The emulsion was spray dried and the resulting dry particles were collected at a 60% yield. This example provides good dissolution, however the total process yield was approximately 17%. That is, Example 2 required two consecutive spray drying steps: one for the drug Z seed particles, and one for the Pulmosphere emulsion. Hence final yield was low. Comparative formulation 123-32-6 (curve labelled with a diamond) is a generally conventional PulmoSphere suspension wherein drug Z particles were suspended in a cosolvent solution of THF and water. However, the suspension was highly acidified in order to achieve (dissolution) and whilst the dissolution profile is good, the resulting particle pH was too low to be usable for pharmaceutical purposes. The final curve (formulation NX, labeled with an “x”) is a micronized crystalline form of drug Z.

### Physical Properties

Lot 123-32-1 (spray-dried powder) made in accordance with embodiments of the present invention, was evaluated for dissolution rate in comparison with crystalline drug Z (in neutral form), as shown by lots 123-32-3 and 123-32-6. FIG. 4 indicates the amount of drug dissolved as a function of time. The dissolution testing was conducted in a simulated lung fluid which included 0.05 molar phosphate, 0.1% tween 80, a pH of 7.4 and temperature of 37° C. All three lots dissolved rapidly to a high plateau. In contrast, the crystalline API (neutral form) dissolves more slowly and reaches a much lower level; at 300 minutes, less than 20% is dissolved. These data indicate that the dissolution rate and solubility of amorphous drug Z, prepared in accordance with the present invention is significantly improved. The SEM image in FIG. 3 shows that formulation 123-32-1 exhibits a desired “honeycomb” structure with interstitial spaces.

### Example 3—Integrated Process Example—Combination of Spinodal Decomposition and PulmoSphere Formulation

At the end of the spinodal decomposition process, drug Z solidifies as amorphous particles suspended in the aqueous co-solvent solution. To utilize spinodal decomposition materials, one usually would go through filtration, drying, and milling steps to obtain a dry powder with desirable particle size. In some embodiments of the PulmoSphere process, an annex suspension is prepared by suspending a poorly soluble drug in water. Because this oftentimes requires starting with a dry, solid drug material, in some embodiments the process may be facilitated by some or all of the steps of filtering, drying, and milling the spinodal decomposition material.

However, in embodiments of the present invention, the spinodal decomposition product materials are advantageously used directly in the PulmoSphere process without further downstream processing. This obviates the need for additional steps must such as filtering, drying, and/or milling. In this embodiment, the annex suspension consists of the spinodal decomposition material suspended in the aqueous cosolvent medium used for spinodal decomposition. This annex may then be mixed with a vehicle emulsion to make a final feedstock for spray drying. This approach is referred to herein as an Integrated Spinodal PulmoSphere (ISP) process.

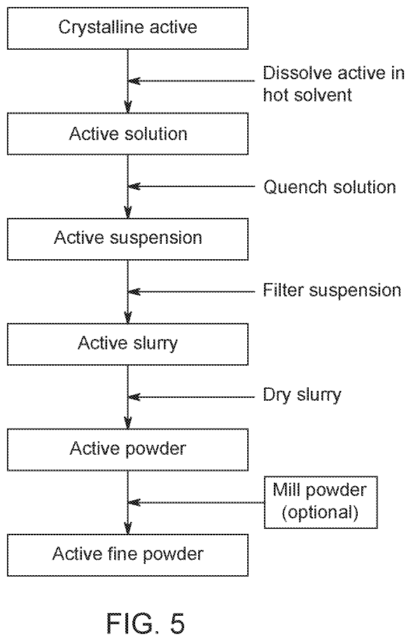

Embodiments of the integrated spinodal PulmoSphere process comprise the direct combination of the spinodal decomposition with PulmoSphere formulation steps, resulting in engineered particles that contain amorphous drug Z in a direct process, that is, wherein there is a consistent process flow, as well minimal or no extraneous process steps. The integrated process has advantages of higher yield and efficiency, as compared to a multi-step approach using particles that have been previously dried. In practice, manufacture of amorphous material by spinodal decomposition may be carried out by a process substantially as shown in FIG. 5. The steps described herein are with reference to both a general and a more specific process. First, the crystalline material such as an API is dissolved in a solvent such as hot THF/water solvent. In the quenching step, the solution is poured into a quenching meeting, for example ice water under agitation to obtain a suspension. In the third step, the suspension is filtered to separate solid from liquid. Then, the solid slurry is dried to remove the residual solvent leaving a dry powder. In some instances, the dry powder may not be sufficiently fine, or have the required particle size distribution, therefore in an optional process step, the size of the initial solid powder material may be reduced by a milling means, such as by jet milling.

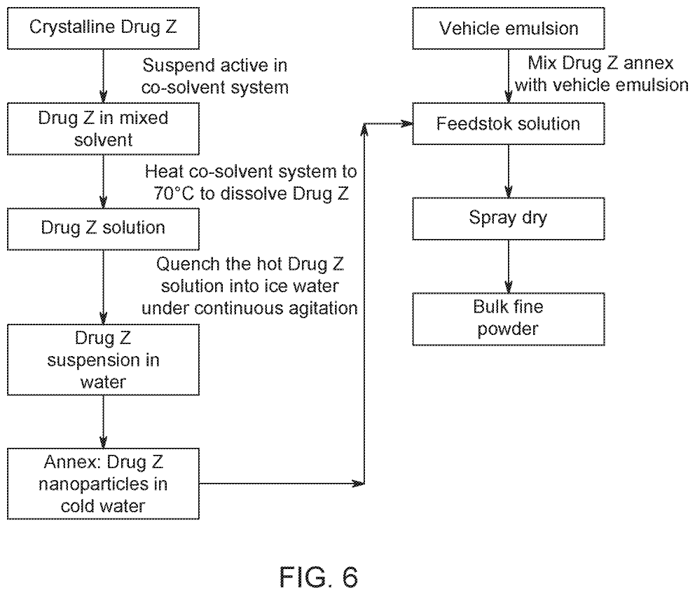

FIG. 6 illustrates an exemplary process whereby an annex drug suspension obtained from spinodal decomposition particle after quenching, which is then mixed directly with emulsion to form the final feedstock. Because the integrated process eliminates the intermediate steps of filtration, drying, and milling, it is faster, reduces yield loss, and results in less chemical degradation. In addition, this approach takes advantage of applying spray-drying technology to produce respirable dry powders in a single unit-operation process.

To streamline the integrated process, direct mixing of annex suspensions with fine emulsions without removing the residual THF solvent could simplify the overall procedure. However, one of the major concerns in formulating a PulmoSphere feedstock is the emulsion stability in the presence of an organic solvent such as THF. It is well known that organic solvents, for example, alcohols such as isopropanol, ethyl alcohol or THF can destabilize emulsions, causing phase separation of PFOB and water. Based on the formulation calculations, the amount of THF in the final feedstock is close to 3% w/w. To study the effects of THF solvent on the emulsion stability, a series of experiments were performed by adding THF at concentrations from 0 to 6% w/w into an emulsion while maintaining the solids content close to that of the feedstock formulation, as shown in Table 1. The emulsion comprised 94% DSPC and 6% CaCl₂). No drug was present. Sample A, which did not contain any THF, is the control. The most convenient way to determine the stability of the emulsion is to measure the emulsion droplet size as a function of time because droplet coalescence or phase separation would result in a change in droplet size. Table 1 shows the results of droplet size of emulsions spiked with various amounts of THF. Comparison of the initial droplet size to that after 24 hours shows that the droplet sizes of PulmoSphere emulsions do not change in the presence of the different levels of THF over 24 hours. Even at 6% w/w THF, which is double the amount that would be used in the integrated spinodal PulmoSphere formulation, the droplet size shows no change after 24 hours. Accordingly, contrary to the conventional teaching that THF can destabilize an emulsion it has been found that at sufficiently low concentrations THF does not adversely impact the emulsion. This result means that in embodiments of the invention, a drug annex and vehicle emulsion may be directly mixed during feedstock preparation.

TABLE 1
Emulsion stability in the presence of THF

| Sample | THF in | Emulsion droplet size ×50, micron | |
|---|---|---|---|
| ID | feedstock, % w/w | t = 0 hour | t = 24 hours |
| A | 0% | 0.27 | 0.25 |
| B | 1% | N/A | 0.27 |
| C | 2% | 0.27 | 0.27 |
| D | 4% | N/A | 0.27 |
| E | 6% | 0.27 | 0.27 |

### Example 4—Polymorphism

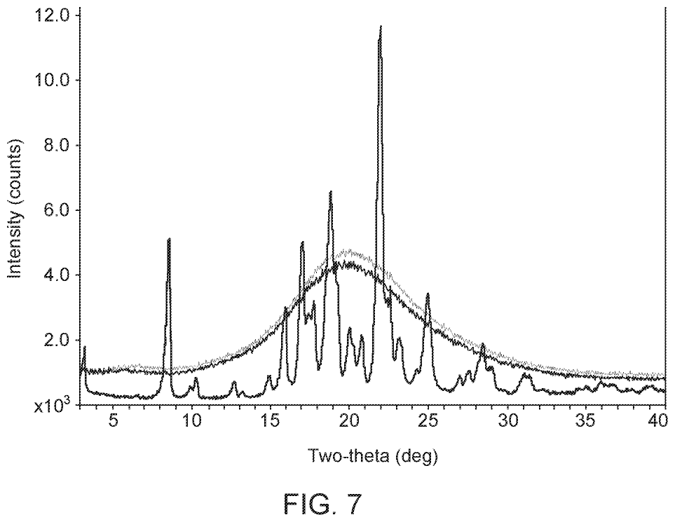

In this Example, it is shown that the material made from a spinodal decomposition process according to embodiments of the invention had been converted to an amorphous form. X-ray powder diffraction (XRPD) was used to confirm that amorphous drug Z was obtained from spinodal decomposition process according to Example 1. The X-ray sample was prepared by centrifuging a suspension manufactured using spinodal decomposition. After decanting the supernatant, the remaining slurry was placed in a vacuum oven at ambient temperature for more than two days to obtain a dry powder for the analysis. FIG. 7 displays X-ray powder diffraction patterns of two lots of drug Z made by a spinodal decomposition process of the present invention, and an original crystalline drug Z. The results show that both spinodal decomposition APIs are amorphous, as evident by a broad diffuse pattern without sharp diffraction peaks; in contrast, crystalline API exhibits a typical pattern with multiple diffraction peaks. As can be seen from the overlay of the spinodal decomposition curves, the resulting materials are nearly identical.

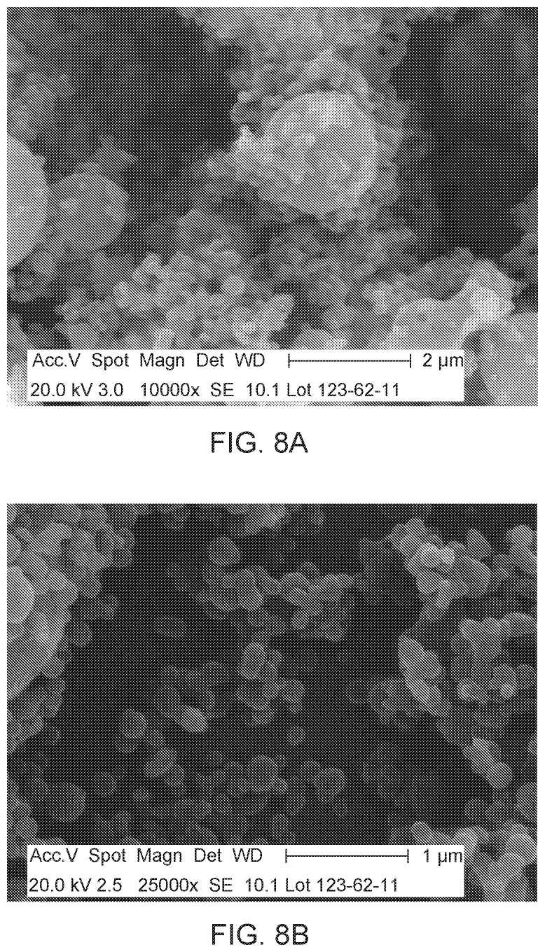

FIGS. 8A-8B show SEM images of drug particles made using a spinodal decomposition process according to Example 1. The FIG. 8A image shows that the majority of the particles are between 200 and 300 nm and the larger particles are one to two microns in size. The higher magnification image of FIG. 8B shows that the particles are fairly uniform in size indicative of a highly ordered phase transformation. It is likely that the smaller particles are the primary particles when phase separation took place in the early stage. After the onset of phase separation, the droplets may have grown by both coalescence and Ostwald ripening, leading to the formation of larger particles as well as aggregates. In embodiments of the invention, some coalescence is potentially beneficial because it facilitates handling of the particles.

### Example 5—Integrated Spinodal PulmoSphere Spray Dried Product

Table 2 lists a series of experiments used to investigate different Integrated spinodal PulmoSphere (ISP) formulations and processes. It was previously noted that phase separation of drug Z during quenching might be an important step for controlling the droplet formation and subsequently particle size of the annex suspension. Each of the examples in the table below utilize embodiments of the process of the present invention as described, for example, in FIG. 6. Formulation components are as noted. In addition, each emulsion originally contained 20% PFOB.

TABLE 2
Integrated Spinodal Decomposition Process (ISP) Development Spray-Drying

| | | | | | DSPC | | |
|---|---|---|---|---|---|---|---|
| | | | Drug | Solid | + | | |
| | | Solvent | Z | conc¹ | CaCl₂ | | Yield |
| Lot # | Formulation | system | % w/w | % w/w | % w/w | Mixing Method | % |
| 123-40-1 | ISP | Water/ | 10% | 3% | 90% | Stir-bar | 60% |
| | | trace THF | | | | | |
| 123-40-2 | ISP | Water/ | 10% | 3% | 90% | Sonication | 72% |
| | | trace THF | | | | | |
| 123-40-3 | ISP | Water/ | 10% | 3% | 90% | High-shear | 70% |
| | | trace THF | | | | mixer | |
| 123-40-6 | ISP | Water/ | 7.4% | 3% | 90% | Stir bar | 62% |
| | | trace THF | | | | | |
| 123-40-7 | ISP | Water/ | 7.4% | 3% | 90% | High shear | 66% |
| | | trace THF | | | | mixer | |

In the table, the first three lots 123-40-1, 123-40-2, and 123-40-3 were of identical formulation composition, but different mixing methods were used in the process. Thus, the effect on droplet formation caused by the flow field of the water phase when introducing the drug Z heated solution into ice water were investigated. C. The mixing approaches were stir-bar (lot 123-40-1), sonication (lot 123-40-2), and T-10 rotor-stat UltraTurrax® high-shear mixer (lot 123-40-3). From a visual assessment of the dispersions, there were no obvious differences noted. After spray drying, powder visual appearance and yields were comparable. Because the upper limit of solubility of drug Z in THF/water at 70° C. is about 10% w/w (THF/water was used in the first three lots) some precipitation on the edge of the container was observed and attributed to solvent evaporation. This is exacerbated when the concentration of drug Z in THF/water is close to its solubility limit. To avoid premature precipitation (due to fast solvent evaporation) at elevated temperature, the concentration of drug Z in THF/water should preferably be somewhat below the active's solubility (approximately 10% w/w). In lots 123-40-6 and 123-40-7, the drug Z concentration in THF/water was reduced to 7.4% to prevent any undesirable precipitation during solution preparation. The mixing methods were stir-bar (lot 6) and T-10 high-shear mixer (lot 123-40-7). The powder yields of these two lots were comparable to those of lots 123-40-1, 123-40-2 and 123-40-3. Based on this study, in some embodiments, the apparatus and shear rates may be used to influence the manufacture of suspensions.

As noted herein, an advantage associated with the integrated spinodal process (ISP) is that final particle yields are higher because, in part, only a single spray drying step is necessary.

### Example 6 Manufacture of Drug Z Inhalation Powder for an Animal PK Study Using ISP Process

A 60 g batch of drug Z inhalation powder was manufactured as described below. To produce the spinodal decomposition material with an amorphous form, the temperature and flow rate of the drug Z solution was controlled during quenching into ice water. The solution was heated to and maintained at 70° C. Flow rate was controlled through the use of a high-accuracy flow control syringe pump was employed. Table 3 illustrates the steps of feedstock preparation with composition information at process intermediates. First drug Z crystals were added to a THF/water co-solvent in a glass vial and then heated to 70° C. After the solution became clear, it was withdrawn from the vial into a 50 cm³ syringe wrapped with heating tape set at 70° C. and placed on a pump rack. Next, the drug Z solution was injected at a flow rate of 5 mL/min into ice water under constant agitation. After the drug Z solution injection was completed, the vehicle emulsion was mixed with the annex suspension to prepare the final feedstock. Table 4 shows the spray-drying conditions of this batch. During the course of spray drying, the feedstock solution was kept at 2-8° C. under continuous agitation.

TABLE 3
Composition at Process Intermediates during Feedstock Preparation

| | | | Solid |
|---|---|---|---|
| | | Medium | concentration |
| Preparation Step | Components | % w/w | % w/w |
| drug Z dissolved in | drug Z | THF/Water | 7.0% |
| co-solvent at 70° C. | | (80/20) | |
| drug Z solution | drug Z | THF/Water | 0.5% |
| quenched | | (5/95) | |
| into ice water | | | |
| Vehicle emulsion | DSPC/CaCl₂, | Water | 4.8% |
| | PFOB drug Z, | | |
| | DSPC/CaCl₂, | | |
| PulmoSphere | PFOB | THF/Water | 3.0% |
| feedstock | | (3/97) | |

TABLE 4
Spray Drying Process Conditions for the Manufacture of
drug Z Powders 123-48.

| | | Atomizer | Drying | Liquid | |
|---|---|---|---|---|---|
| | | gas | gas | feed | Collector |
| Inlet | Outlet | flow rate, | flow rate, | rate, | temp, |
| temp, ° C. | temp, ° C. | L/min | L/min | mL/min | ° C. |
| 112 | 70 | 25 | 600 | 10 | 60 |

### Analytical Results of Example 6

Table 5 shows the physicochemical properties of the spray-dried powders of Example 6. The primary particle size is 2.9 μm which is within the targeted range, 2.5-3.5 μm. The bulk and tapped density is also within the preferred range for typical PulmoSphere particles. The drug content is 9.2% which is close to he targeted value of 10% w/w. This may have been due to some of the drug particles being carried away by the effluent gas during cyclone collection. The yield was 86%.

TABLE 5
Integrated Process Spray-dried Powder
Physicochemical Properties

| Physical Property | Value |
|---|---|
| Primary particle size, micron | 2.9 |
| Bulk density, g/cm³ | 0.048 |
| Tapped density, g/cm³ | 0.069 |
| Water content, % w/w | 2.9 |
| Drug content, % w/w | 9.2 |
| Spray drying yield, % | 86 |

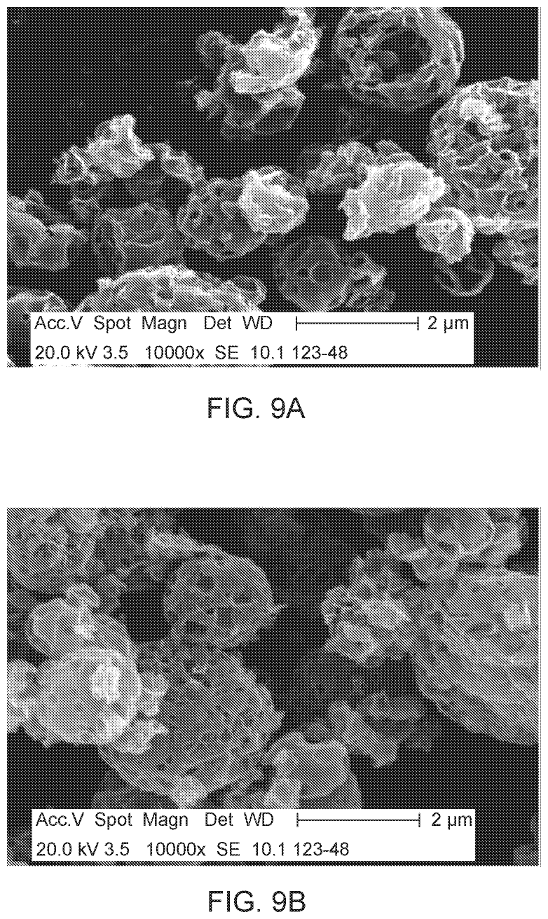

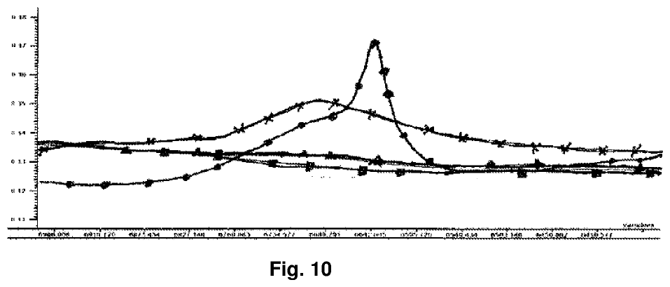

SEM images, shown in FIG. 9A-9B, demonstrate the characteristic porous PulmoSphere morphology of the particles of Example 6.

Because XRPD analysis might not be sensitive enough to detect any crystalline drug Z in a formulation with such a low drug loading (10% w/w), a NIR spectroscopy method was employed.

FIG. 10 shows the diffuse reflectance spectra of: (i) non-spinodal crystalline neat drug (the curve with single sharp peak, labelled with dots); (ii) spinodal decomposition neat drug made in accord with Example 1 (the next highest curve labelled with “x”s); (iii) a spray-dried drug powder made according to a spinodal decomposition/engineered particle process of the present invention (Example 2—the intermediated curve labelled with triangles); and (iv) a placebo (non-spinodal, no active PulmoSphere powder.—the lowest curve labelled with squares). It can be seen that the spray-dried powder made using a spinodal process of the present invention is amorphous after spray drying (see the broad, diffuse peak between wave numbers 6595 and 6827 cm⁻¹). The graph also shows that the placebo curve is slightly distinct from the PulmoSphere formulated spinodal process drug Z. The results show that drug Z spray-dried powder made from integrated spinodal PulmoSphere process is amorphous.

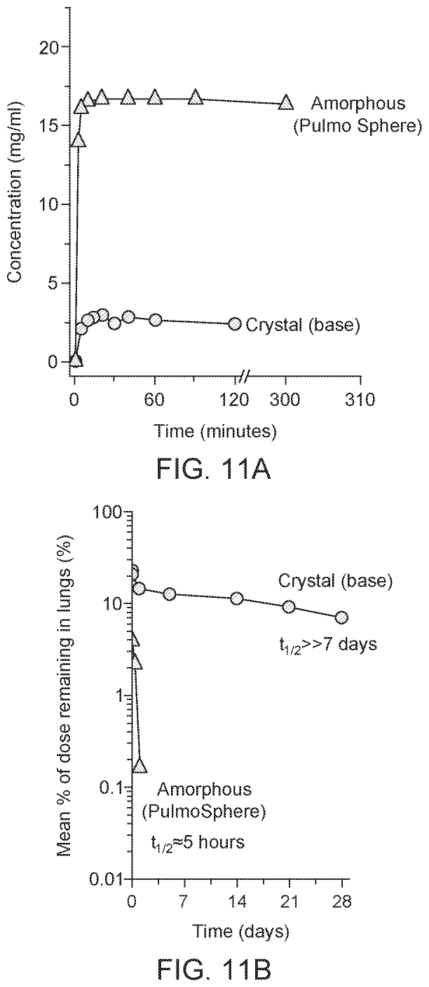

Amorphous nanostructured drugs provide for desirable physical properties that enable advantageous in vivo performance, as exemplified in FIGS. 11A and 11B. FIG. 11A shows a comparison of the in vitro dissolution profiles of crystalline drug Z as well as an amorphous, nanostructured form manufactured using the process described herein (Example 2). In comparison to the amorphous form, the crystalline form has a markedly lower dissolution rate, as given by the shallower initial slope during the first few minutes of dissolution. The crystal also has a lower apparent solubility, as given by its lower plateau in the dissolution profile. Even for periods as long as five hours, the amorphous form has a higher solubility. In this case, the ratio of the amorphous and crystalline solubilities—the solubility advantage is approximately 6. FIG. 11B shows pharmacokinetic results, as given by the time dependence of the measured lung concentrations following intra-tracheal delivery of these different solid-state forms to rats. The half-life of each form is indicated on the plot. The crystalline form has an exceedingly long half-life, more than 7 days, which raises concerns for accumulation of drug in the lungs and, potentially undesirable local toxicological issues (e.g., irritation of the lung epithelium). In contrast, the half-life of the amorphous, nanostructured form is more than 30 times shorter (about 5 hours), indicating that dissolution and absorption is faster for this form. Thus, the data shown in FIGS. 11A and B demonstrate a causal link between the physical form and biopharmaceutical performance.

Having now fully described this invention, it will be understood to those of ordinary skill in the art that the methods and formulations of the present invention can be carried out with a wide and equivalent range of conditions, formulations, and other parameters without departing from the scope of the invention or any embodiments thereof.

All patents and publications cited herein are hereby fully incorporated by reference in their entirety. The citation of any publication is for its disclosure prior to the filing date and should not be construed as an admission that such publication is prior art.

1. A method for preparing an amorphous nanostructured active material comprising
preparing a suspension or dispersion of a poorly water-soluble active material in a solvent, wherein the solvent is selected to solubilize a desired quantity of the material upon heating, and wherein the suspension or dispersion comprises the active material and solvent;
heating said suspension or dispersion to a temperature sufficient to dissolve the active material to yield a solution;
quenching the solution, by metering into a temperature-controlled quenching medium while mixing using high-shear, resulting in a spontaneous liquid-liquid phase separation, yielding a first active material-rich phase and a second solvent-rich phase wherein solid amorphous particles of active material precipitate from the first active material-rich phase; and
collecting said solid amorphous particles.

2. The method of claim 1 wherein said poorly water-soluble active material has a percentage dissolved of less than about 20% and solid amorphous particles resulting have a percentage dissolved of at least about 60%.

3. The method of claim 1 wherein said solid amorphous particles resulting have a solubility of at least two times greater than said poorly water-soluble active material.

4. The method of claim 1 wherein said solid amorphous particles resulting have a percentage dissolved of at least 80%.

5. The method of claim 1 wherein said solid amorphous particles are nanoscale and have a honeycomb morphology with interstitial spaces.

6. The method of claim 5 wherein said solid amorphous particles have a primary particle size range of 100-500 nanometers.

7. The method of claim 1 wherein allowing the quenched formulation is allowed to dwell to permit coarsening of drug-rich droplets and precipitation thereof into solid particles.

8. The method of claim 1 wherein the quenching is performed under a defined sink condition.

9. The method of claim 8 wherein quenching comprises immersion in an ice water bath.

10. The method of claim 8 wherein the defined sink condition comprises a substantially constant quench temperature environment.

11. The method of claim 1 wherein the solvent comprises water.

12. The method of claim 1 wherein the solvent comprises a two-component system comprising water and a mater-miscible co-solvent.

13. The method of claim 12 wherein the two-component solvent system comprises water and THF.

14. The method of claim 1 wherein said mixing Damkohler number is less than 1.

15. A particulate product made by the method of claim 1.

16. A method for preparing an amorphous nanostructured pharmaceutical material comprising
preparing a suspension or dispersion of a poorly water-soluble active pharmaceutical ingredient in a solvent, wherein the suspension or dispersion comprises the active and solvent;
heating said suspension or dispersion to a temperature sufficient to substantially dissolve the active pharmaceutical ingredient to yield a solution;
quenching the solution, by metering into a temperature-controlled quenching medium while mixing using high-shear, resulting in a spontaneous liquid-liquid phase separation, yielding a first active material-rich phase and a second solvent-rich phase wherein solid particles of amorphous active material precipitate from the first active material-rich phase; and
collecting said solid amorphous particles.

17. The method of claim 16 wherein allowing the quenched formulation is allowed to dwell to permit coarsening of active-rich droplets and precipitation thereof into solid particles.

18. The method of claim 16 wherein the active pharmaceutical ingredient comprises two or more active pharmaceutical ingredients.

19. A soluble amorphous material prepared by the process of claim 16.

20. The soluble amorphous material of claim 19 characterized in that it is excipient free.

21. A method for preparing a pharmaceutical powder comprising
preparing a suspension or dispersion of a poorly-water soluble active pharmaceutical ingredient in a solvent, wherein the suspension or dispersion consists of only the material and solvent;
heating said suspension or dispersion to a temperature sufficient to dissolve the active pharmaceutical ingredient to yield a solution;
quenching the solution, by metering into a temperature-controlled quenching medium while mixing using high-shear, resulting in a spontaneous liquid-liquid phase separation, yielding a first active-rich phase and a second solvent-rich phase; and
allowing the quenched formulation to dwell to permit coarsening of active-rich droplets and precipitation thereof into solid nanoparticles of substantially pure active pharmaceutical ingredient in amorphous form;
collecting said solid particles;
preparing an emulsion of the solid nanoparticles of active pharmaceutical ingredient in a solvent or suspending agent, together with a phospholipid to yield a feedstock; and
spray drying feedstock to yield nanoparticles of active pharmaceutical ingredient with a honeycomb morphology with interstitial spaces.

22. A powder prepared by the method of claim 21

23. The powder of claim 22 suitable for pulmonary administration.

24. The powder of claim 22 suitable for oral administration.
